(19) United States
(12) Patent Application Publication
(10) Pub. No.: US 2004/0116815 A1
(43) Pub. Date: Jun. 17, 2004

(54) Automatic sound blood measurement device

(76) Inventors: Wen-Feng Yang, Hsinchu (TW); Cheng-Cheng Lee, Taipei (TW); Kuo-Pin Chen, Hsinchu (TW)

Correspondence Address: LOWE HAUPTMAN GOPSTEIN GILMAN & BERNER, LLP, Suite 310, 1700 Diagonal Road, Alexandria, VA, 22314, US

(21) Appl. No.: 10/317,071

(22) Filed: Dec. 12, 2002

(51) Int. Cl. A61B 5/02

(52) U.S. Cl. 600/485

(57) ABSTRACT

The present invention is a automatic sound blood measurement device, it output a blood sense signal to a micro-computer CPU to operate from a blood pressure and voltage signal transform interface circuit; then, the operation result signal of said micro-computer CPU through a sound mixer to generate a analog mix signal, and inform the patient by sound broadcasting method through a speaker, and in the mean time it also can show up on the display screen by digital method, and the patient can easily read the blood pressure and related function (such as systolic blood pressure, diastolic blood pressure, heart beating, and auxiliary medical sound reading) messages by hearing and seeing.

### BACKGROUND OF THE INVENTION

1. Field of the Invention

The invention relates to a automatic sound blood measurement device, it output a blood sense signal to a micro-computer CPU to operate from a blood pressure and voltage signal transform interface circuit; then, the operation result signal of said micro-computer CPU through a sound mixer to generate a analog mix signal, and inform the patient by sound broadcasting method through a speaker, and in the mean time it also can show up on the display screen by digital method, then, the tester can easily read the blood pressure and related function (such as systolic blood pressure, diastolic blood pressure, heart beating, and auxiliary medical sound reading) messages by hearing and seeing, and it is for the purposes of convenient and worm used for blind, hard walker, dark environment used, and under the condition of lacking medical persons.

2. Description of Related Art

The conventional technology of blood pressure measurement device, one kind is to read the analog mmHg numeric of blood pressure by seeing, and another kind is digital display, when the tester turn on the machine, the motor connects the air compressor to push the air to the bandage on the arm, according to the pressure sensor on the bandage, output the blood pressure signal to the computer CPU to operate, and then show up the operation result on the display screen, but it still has the following defects:

1. It is difficult to read for old man, even more, it is easily to error reading.

2. The Diabetes patient has the trouble of eye seeing, can not read and judge by his own.

3. The blind must go to hospital or need others help.

4. When read the blood pressure numeric needs sufficient light.

5. It is un-convenient to read the measurements in the night.

6. The prior blood pressure machine can not satisfy and attract the youth.

7. Under emergency and the condition of helpless, could be error judge because of the trouble of eye seeing.

According to above defects, we design the present invention:

The first object of the present invention is to provide a automatic sound blood pressure device which can broadcast report by sound for eye weak, blind, work un-convenient, you can measure it by yourself, and avoiding the patient tester need go to hospital or under the condition of no others help.

The second object of the present invention is to provide a automatic sound blood pressure device which can satisfy the youth and let them have the habit of measuring blood pressure.

### SUMMARY OF THE INVENTION

Base on the idea described above, the present invention is to provide a automatic sound blood pressure device; It is a automatic sound blood measurement device, comprising: a blood pressure and voltage signal transform interface circuit, it transforms sense signal of analog blood pressure to digital blood pressure signal; a micro-computer, it transport the output digital blood pressure signal of said blood pressure and voltage signal transform interface circuit to said micro-computer CPU to operate; a sound mixer, it transform the operating result of said microcomputer CPU to sound mixer signal; and a speaker, it output the sound mixer signal of said sound mixer to be a amplify signal by sound broadcasting.

Base on the idea described above, wherein said blood pressure and voltage signal transform interface circuit is a analog to digital transform interface circuit (A/D converter).

Base on the idea described above, wherein said microcomputer CPU also can output the operation result digital signal to display by digital method.

Base on the idea described above, wherein said micro-computer CPU can according to different needs to transform the operation result digital signal to be different languages digital signal, then through said sound mixer can become analog sound mix signal of different languages.

Because of the progressive of present society, the nations income is more higher, and people give more attention on the subject of health care, therefore it is the most important subject in life time about disease preservation, even more important than the cured medical action, so called, “The preservation is prior to cure”. It is a very important concept of health care about the preservation for modern people and future medical, such as the disease of high blood pressure is more and more, not just western countries, even more like China, east south Asia, south America, because the life style of Asian people is more and more affects by the west civilization, cause those diseases are more and more popular in Asia, and pay more attention to the preservation and cure of high blood pressure, such as the high blood pressure patient are increase one and half million people every year. The improve countries people love wine and coffee, and the related search about coffee by John Hopkins university Dr. Michael J. Klag in America, if everyday drink five cups of coffee, last two months, it will cause blood pressure increase, the systolic blood pressure is up 2.4 mmHg average, and the diastolic blood pressure is up 1.2 mmHg, and it is more obvious affects to the youth than to the old man, we still knew the reason why. At present, it is more and more lower of age of high blood pressure population, it is not just for the old man, such as there are report about the sudden death of junior high school students, in addition to the equivalent eating, hold regular exercise, steady emotion, and keep the habit of blood pressure measurement, for above reasons, so we use automatic sound blood pressure devise to measure blood pressure to solve the problems of trouble and error reading. Because the problem of old man is more and more serious, the concepts of health care, in future, the medical market is focus the device of preservation and DIY measurement, and the automatic sound blood pressure device is the future guide direction of government.

The present invention of special model of automatic sound blood pressure device is a modern and self test product, concluding the characteristics as below:

1. humanistic: it is broadcasting by sound, it is a house living and self test product, it is a wormer used product than conventional prior blood pressure machine.

2. life: it is more convenient for users, it is by means of sound broadcasting in time, it is easily to read and it do not need to read by eye seeing.

3. technologic: it output a blood sense signal to micro-computer CPU to operate from a blood pressure and voltage signal transform interface circuit; then, the operation result signal of micro-computer CPU through a sound mixer to generate a analog mix signal, and inform the patient by sound broadcasting method through a speaker, and in the mean time it also can show up on the display screen by digital method.

4. novelty: the common conventional prior art is a eye seeing type blood pressure machine, so far, there is no sound broadcasting type blood pressure machine in the market at present.

5. creation: the present invention is a “talking blood pressure machine”, and it is easier to communicate with the users of the special model automatic sound blood pressure device.

Base on the idea described above, the inventor according to several years experience of R&D and much similar to market, so providing the present improved invention” talking blood pressure machine”, and the major speciality is to solve the problem of without the function of automatic sound broadcasting, conclusion, the characteristic of the modern self test product described above is a very important and much influence to human society, so, the inventor applies this invention.

### BRIEF DESCRIPTION OF THE DRAWING

The invention will be better understood and objects other than those set forth above will become apparent when consideration is given to the following detailed description thereof. Such description makes reference to the annexed drawings wherein:

FIG. 1 is a block diagram of the prior art.

FIG. 2 is a block diagram of the present invention.

### DETAILED DESCRIPTION OF PREFERRED EMBODIMENT

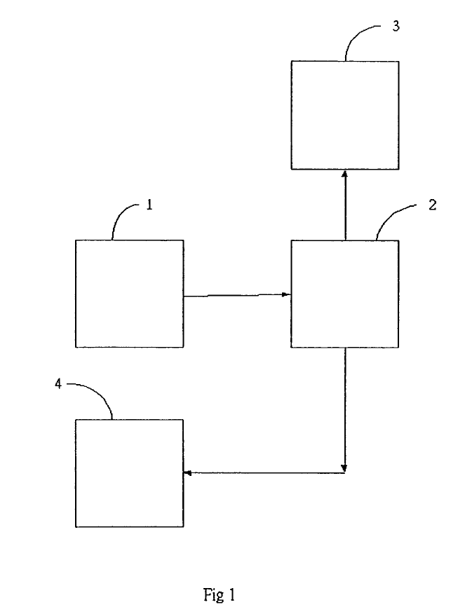

Please refer to FIG. 1, which is a block diagram of the prior art. Firstly, when conventional prior blood pressure device is ON, said blood pressure and voltage signal transform interface circuit 1 transforms the analog blood pressure signal of user to a digital blood pressure signal, and then output to said computer 2 CPU to operate, in the mean time, said computer 2 send a control signal to motor air compressor 4 to work, after, said computer 2 let the operation result show up on the display screen by digit.

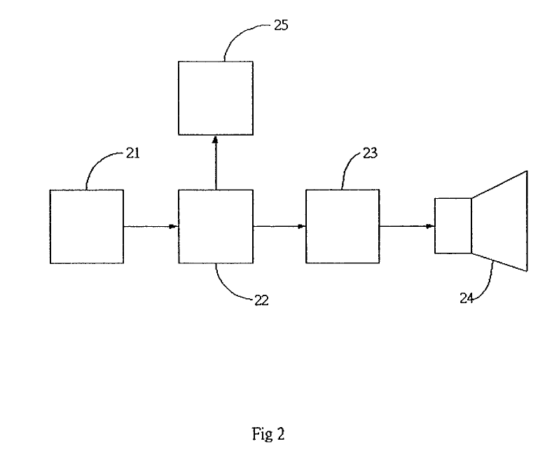

Please refer to FIG. 2, which is a block diagram of the present invention., Firstly, when the device is ON, said blood pressure and voltage signal transform interface circuit 21 transforms the analog blood pressure signal of user to a digital blood pressure signal, it output a blood sense signal to said micro-computer 22 CPU to operate; and then, the operation result signal of said micro-computer 22 CPU through a sound mixer 23 to generate a analog mix signal, and to inform the patient by sound broadcasting method through a speaker 24, therefore, the patient can easily read the blood pressure and related function (such as systolic blood pressure, diastolic blood pressure, heart beating, and auxiliary medical sound reading) messages by hearing and seeing, and in the mean time, it also can show up on the display screen by digit.

The preferred embodiment described above is the better ones, which is to explain but limit the scope of the present invention; the scope of the present invention is defined by the claims described as follow. The variations and modifications according to the claims of the present invention should be included by the present invention.

What is claimed is:

1. A automatic sound blood measurement device, comprising:
a blood pressure and voltage signal transform interface circuit, it transforms sense signal of analog blood pressure to digital blood pressure signal;
a micro-computer, it transport the output digital blood pressure signal of said blood pressure and voltage signal transform interface circuit to said micro-computer CPU to operate;
a sound mixer, it transforms the operating result of said microcomputer CPU to said sound mixer signal; and
a speaker, it output sound mixer signal of said sound mixer to be a amplify signal through said speaker by sound broadcasting.

2. The automatic sound blood measurement device of claim 1, wherein said blood pressure and voltage signal transform interface circuit is a analog to digital transform interface circuit (A/D converter).

3. The automatic sound blood measurement device of claim 1, wherein said micro-computer also can output the digital signal of said operation result to display by digital method.

4. The automatic sound blood measurement device of claim 1, wherein said micro-computer CPU can according to different needs to transform the operation result digital signal to be different languages digital signal, then through said sound mixer can become analog sound mix signal of different languages.
